(19) United States
(12) Patent Application Publication
(10) Pub. No.: US 2021/0370134 A1
(43) Pub. Date: Dec. 2, 2021

(54) LOAD SENSOR DEVICE SYSTEM FOR MEASURING AND TRACKING EXERCISE STRENGTH AND EFFORT

(71) Applicant: ASEEM GUPTA, DUBAI (AE)

(72) Inventor: ASEEM GUPTA, DUBAI (AE)

(21) Appl. No.: 17/327,769

(22) Filed: May 24, 2021

Related U.S. Application Data
(60) Provisional application No. 62/704,740, filed on May 26, 2020.

(51) Int. Cl. A63B 24/00; A63B 71/06

(52) U.S. Cl. CPC A63B 24/0062; A63B 24/0006; A63B 24/0087; A63B 2024/0093; A63B 2024/0065; A63B 2024/0068; A63B 2024/0009; A63B 71/0622

(57) ABSTRACT

This invention relates to a load sensor device system, and more particularly to a load sensor device system for measuring physical exercise strength and effort, that provides load measurements for effort load deployed while a person performs physical fitness exercises using extensible functional resistance exercise apparatus and provides real time load data that can be tracked to demonstrate effectiveness of exercise training over a period of time using the sensor device and system.

This is the complete specification of provisional filed U.S. patent application Ser. No. 62/704740 (under 35 USC 111(b)), which claims priority to U.S. provisional Patent Application 62/704740 filed May 26, 2020. The present non-provisional application has the same inventor as the aforementioned application.

### FIELD OF INVENTION

This invention relates to a load sensor device system, and more particularly to a load sensor device system for measuring physical exercise strength and effort, that provides load measurements for effort load deployed while a person performs physical fitness exercises using extensible functional resistance exercise apparatus and provides real time load data that can be tracked to demonstrate effectiveness of exercise training over a period of time using the sensor device and system.

### BACKGROUND OF THE INVENTION

Resistance training as a form of fitness exercise is an established method for using body weight to perform functional fitness training using passive extensible cable, wire, rope, elastic and mechanical resistance exercise equipment for cross fitness training. The commonly used conventional apparatus is a passive extensible apparatus which provides resistance to a user for loading body weight on the passive extensible cable, wire, rope, elastic and mechanical resistance exercise equipment which are attached to a rigid support on one end and to the body on the other end via a grip handle or hook mechanism that is attached to different body parts. The passive extensible cable, wire, rope, elastic and mechanical resistance exercise equipment is pre-loaded using variable load or rated load based on the type of extensibility being used, such as physical dead weights as well as add on resistance by a plurality of extensible bands or ropes. The existing systems provide functionality but fail to provide a mechanism to measure and record the strength and effort deployed by a user while using such passive functional apparatus that provides resistance in response to loading the body weight on extensible wire, cables or elastic bands.

Hence, there is the need for an invention that provides the passive extensible resistance training equipment the ability to measure, record and track with the objective of demonstrating the effectiveness of the exercise training over a period.

In order to overcome prior art, the present invention addresses specifically the ability to measure, record and track the body weight load effort deployed by revealing a novel sensor device, which, when it is attached to passive extensible cable, wire, rope, elastic and mechanical resistance exercise equipment, provides such a measurement as a novel solution.

### SUMMARY OF THE INVENTION

Accordingly, a load sensor device for measuring and tracking exercise strength and effort is provided. The device has ability to measure, record and track the body weight load effort. The load sensor device comprises a body load sensing device (100) embedded with a miniature tension-compression load sensor element (200), characterized in that a load sensor processor (210) and a load sensor unit (250) abbreviated inside two representative embodiments, in one, a rigid enclosure(120) and, in another, a flexible enclosure (130) which attach to representative forms of extendible and rigid resistance exercise apparatus (300) using anchor buckles (150).

The present invention provides a method of operating the load sensor device using a smart watch application (450), a smartphone application (460) managed using a cloud platform (410)

### DESCRIPTION OF THE DRAWINGS

It is to be noted, however, that the appended drawings illustrate only typical embodiments of this invention and are therefore not to be considered for limiting of its scope, for the invention may admit to other equally effective embodiments.

FIG. 1 reveals a perspective of the invention showing a rigid enclosure connected with flexible bands and anchor hooks on two sides.

FIG. 2 reveals a perspective of the invention showing a flexible textile enclosure connected with flexible bands and anchor hooks on two sides.

FIG. 3 reveals a representation of the load sensor element with its electronic component and electromechanical components bonded together.

FIG. 4 reveals the load sensor processor component of the load sensor element.

FIG. 5 reveals the load sensor unit component of the load sensor element.

FIG. 6A reveals an exploded view of the device revealed in FIG. 1.

FIG. 6B reveals an exploded view of the device as revealed in FIG. 2.

FIG. 7A reveals a hand grip.

FIG. 7B reveals a multiple corded resistance band apparatus.

FIG. 7C reveals a single cord resistance band apparatus.

FIG. 7D reveals an extensible resistance band.

FIG. 7E reveals a set of non-extensible resistance training straps with the device embodiments shown in FIG. 1 and FIG. 2 as attached to the strap apparatus.

FIG. 8 reveals a technical hierarchy of the system layers.

FIG. 9 reveal system data and command flows between the device, the smartphone application and the cloud platform that make up a functional system

FIG. 10A reveals an example of a human figure using extensible resistance training apparatus showing the use and location of the invention.

FIG. 10B reveals an example of a human figure using a rigid strap resistance training apparatus showing the use and location of the invention.

FIG. 10C reveals a representation of the user interface for the FIG. 10A.

FIG. 10 D reveals a representation of the user interface for the FIG. 10B.

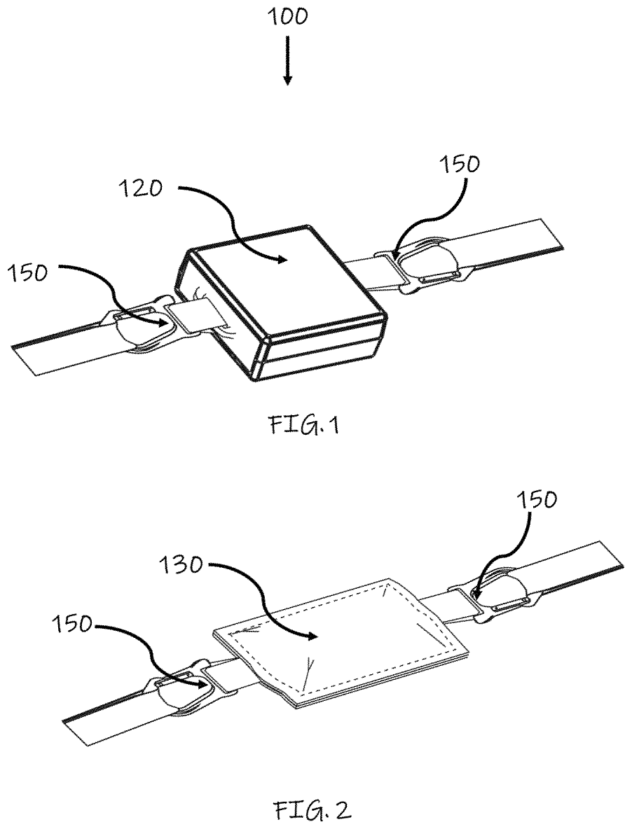

### DETAILED DESCRIPTION OF THE INVENTION:

The following detailed description refers to the accompanying drawings, which illustrate specific embodiments. Other embodiments having different structures and operation do not depart from the scope of the present disclosure.

The present invention relates to a body load sensing device representation (100) embedded with a miniature tension-compression load sensor element (200), comprising of a load sensor processor (210) and a load sensor unit (250) abbreviated inside two representative embodiments, in one, a rigid enclosure(120) and in the other a flexible enclosure (130) which attach to representative forms of extendible and rigid resistance exercise apparatus (300) using anchor buckles (150), and a method of operating it using a smart watch app (450), a smartphone app (460) managed using a cloud platform (410).

The resistance exercise load sensing device (100) comprises of an enclosed load sensor element (200), not limited to this specific aspect in form or scope of enclosures in the shown embodiments, of a rigid enclosure (120) comprising of a plastic case and a flexible enclosure (130) which can be stitched, or adhesion molded. The resistance exercise load sensing device (100) shown in representation has anchor buckles (150) made of sturdy material construction to withstand high loads which are attached to the load sensor element (200) on either side at the anchor location (260)—FIG. 5. The load sensing device (100) not limited to its shown embodiments (120, 130) can attach to a plurality of resistance exercise apparatus (300) options such as, but not limited to, grip handles, straps, extendible cords shown in Sheet 300A and Sheet 300B. The attachment to the resistance exercise apparatus (300) is via the anchor buckles (150) as shown (260)—FIG. 5, however, any other type of attachment mechanism is possible to tension the load sensor unit (210).

As one skilled in the art would realize that various other shapes and sizes could be created with adaptations and added functionalities.

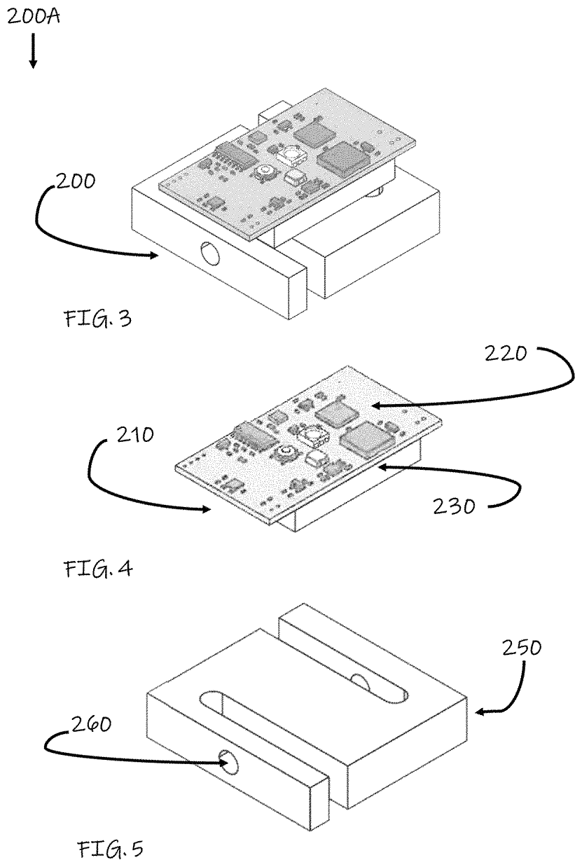

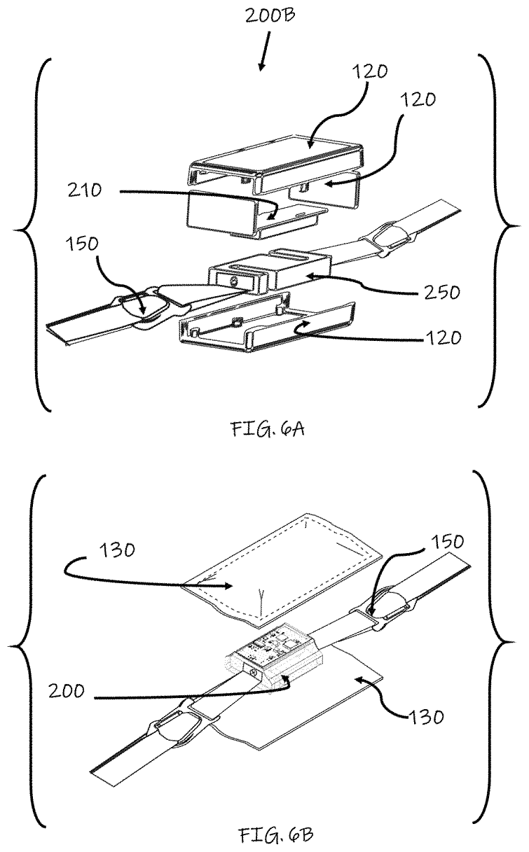

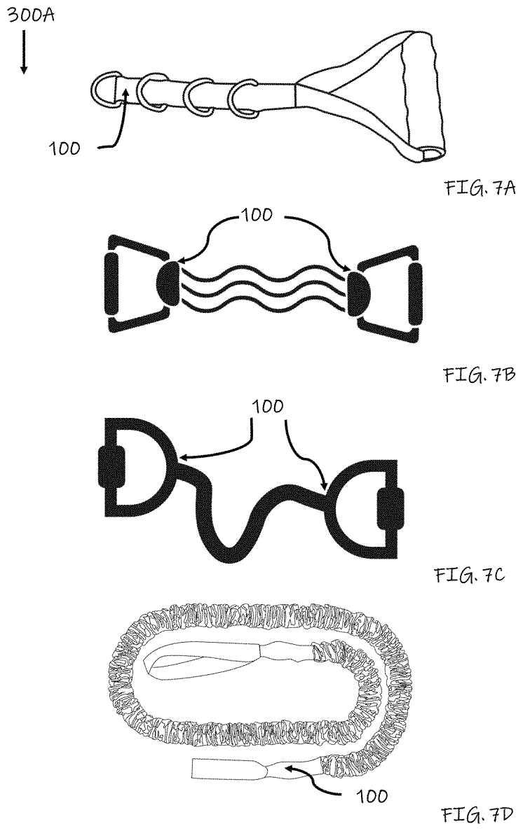

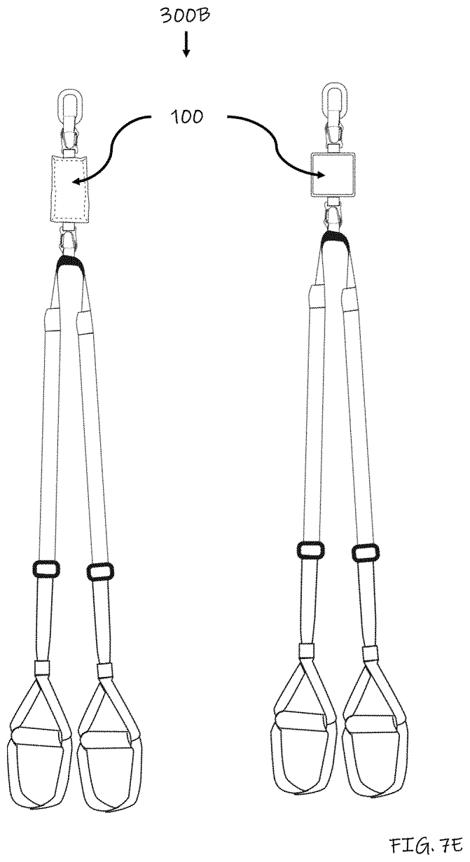

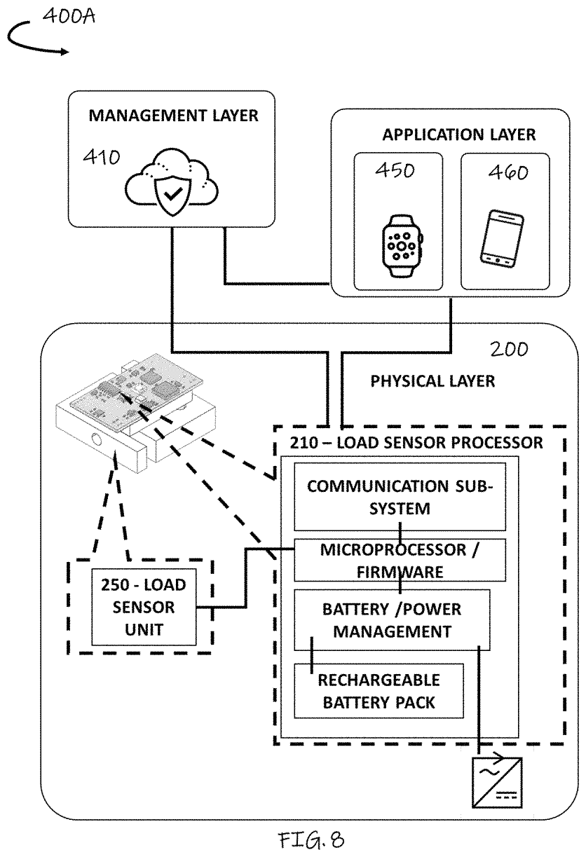

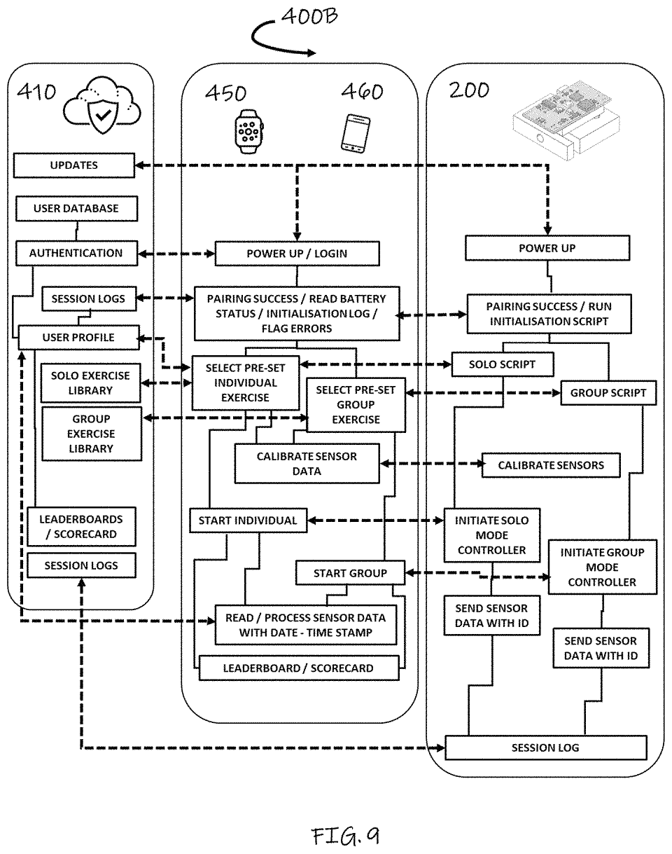

In its most generic format, the load sensor element is crafted to include a set of components (FIG. 3, FIG. 4, FIG. 5) which make up a functional battery powered wireless tension-compression load sensor with the ability to exchange data with a compatible application (450, 460) and a management cloud (405).

Drawings Page 3/9 Ref 200A, reveals an exploded view of the two representative embodiments, in one a rigid enclosure (120) (FIG. 6A) and in the other a flexible enclosure (130)(FIG. 6B) but not limited to be the only aspects of the invention. The elements and components of an embodiment of the invention may be physically, functionally and logically implemented in any suitable way.

How the Components Connect and Interact

Drawings Page 4/9 Ref 300A, reveals attachment position for resistance exercise load sensing device (100) on different embodiments of known extensible resistance training apparatus.

Drawings Page 5/9 Ref 300B, reveals attachment position for resistance exercise load sensing device (100) on an embodiment of known non extensible strap-based resistance training apparatus.

Drawings Page 6/9 Ref 400A reveals the technical architecture of the system (FIG. 8) showing 3 logical layers, the physical layer (200), the application layer and the management layer. The physical layer is comprised of the load sensor processor(210)which is equipped with wireless connectivity, not limited to the using WIFI, BLE, RF, IR communication technologies, for interaction with the application layer which is comprised of the smartwatch app (450), smartphone app (460) and the management layer which is comprised of a secure cloud platform (410).

The load sensor unit (250) is attached to the load sensor processor (210) by wires (not shown) and provides the tension-compression raw data readings which are processed within the firmware of the load sensor processor (210) as meaningful data and exchanged via the wireless communication module within the load sensor processor (210) with the user's smartwatch app (450), user's smartphone app (460) and a secure cloud platform (410). The load sensor element (200) is powered by a rechargeable battery (FIG. 8).

A single load sensor element (200) or a plurality of load sensor elements may be deployed within the resistance training apparatus (300). The smartwatch app (450), smartphone app (460) and a secure cloud platform (410) are capable of processing data from a single or multiple load sensor elements' attached to a user's resistance training apparatus and the proprietary algorithm logic processes, stores and displays the load readings and tracking information visually with predictive logic over a period of time and graphical data analysis (not shown).

Operation of the Invention

The present invention relates to a body load sensing device representation (100) embedded with a miniature tension compression load cell sensor element (200), comprising of a load sensor processor (210) and a load sensor unit (250) abbreviated inside two representative embodiments, in one a rigid enclosure (120) and in the other a flexible enclosure (130) which attach to representative forms of extendible and rigid resistance exercise apparatus (300) using anchor buckles (150) and a method of operating it using a smart watch app (450), a smartphone app (460) managed using a cloud platform (410).

Exercise activity may be performed at all types of indoor and outdoor locations for users while performing and participating in individual and group functional training using extensible and non-extensible resistance training apparatus.

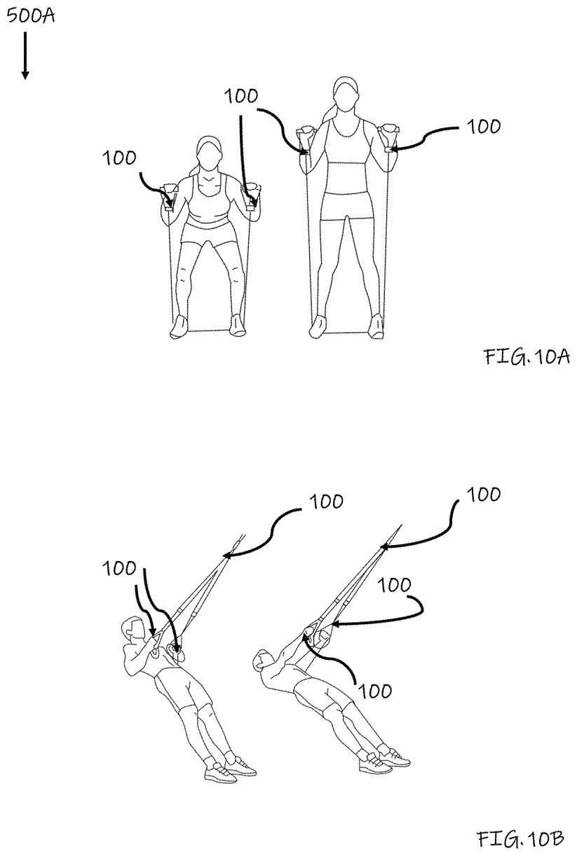

Drawings Page 7/9 Ref 400B (FIG. 9) provides the individual and cross functional operational flow between the physical layer (load sensor element 200), application layer (smart watch app 450/smartphone app 460) and the management layer (cloud platform 410). On powering up the load sensor element and initiating the application an initialization is performed which checks the battery status and runs the initialization script on the load sensor processor (210). On success of the pairing the smartphone app (460) can assign settings to the load sensor element (200) and create a user login account with the managed cloud platform (410). This login account can identify the specific user account with the specific device ID to ensure the readings are stored within the user profile to provide tracking analytics.

The smartwatch app (450) pairs with the smartphone app (460) to provide the user a graphical, visual and textual display of the readings while the user participates in the resistance exercise using the resistance exercise apparatus (300).

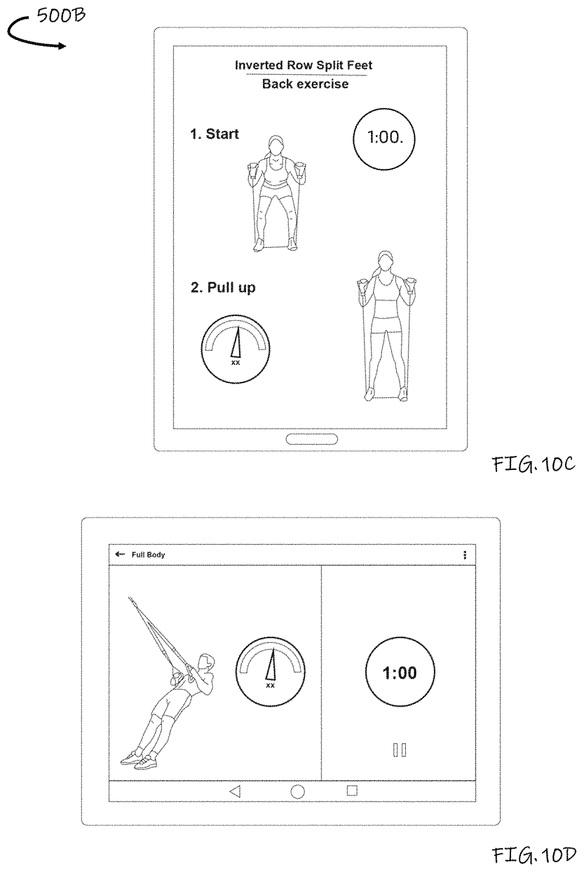

Drawings Page 8/9 Ref 500A (FIG. 10A) shows an illustrated example of a human figure doing an exercise with an extensible resistance exercise apparatus equipped with the invention (100). The load sensor element (200) abbreviated within the invention (100) streams the real-time readings as they change to the connected application and management layer for storage, processing and visualization.

Drawings Page 8/9 Ref 500A (FIG. 10B) shows an illustrated example of a human figure doing an exercise with a non-extensible strap resistance exercise apparatus equipped with the invention (100). The load sensor element (200) abbreviated within the invention (100) streams the real-time readings as they change to the connected application and management layer for storage, processing and visualization.

Drawings Page 9/9 Ref 500B (FIG. 10C) shows a representation of an illustrated example of the visualization of the real-time readings on an application user interface with time and load displayed together with an animated visual of the same exercise being performed in FIG. 10A.

Drawings Page 9/9 Ref 500B (FIG. 10D) shows a representation of an illustrated example of the visualization of the real-time readings on an application user interface with time and load displayed together with an animated visual of the same exercise being performed in FIG. 10B.

The elements and components of the operational embodiments are revealed to demonstrate the functionality of the invention which may be physically, functionally and logically implemented in any suitable way as well as may possibly be advantageously combined with other smartphone, smartwatch apps using application interfaces as well as provide data interchange with other cloud platforms.

Alternative Embodiments

In another embodiment, load sensor element (200) comprising of a load sensor processor (210) and a load sensor unit (250) (FIG. 3), can be abbreviated in any other type of functional equipment that produces tension—compression to provide real time data readings. The open communication protocols used within the load sensor processor provide the ability to stream the data readings in open or proprietary data interchange formats making it extremely versatile.

In another embodiment, the smartphone app (460) when connected to the load sensor device (200) allows users to vary the timing of the exercise, the cycles as well as the reps to suit their preferred fitness level which allows the system be useful for people of all ages and fitness levels and provides tracking information of gain or loss in strength while performing the same exercise over a period of time.

In yet another embodiment the load sensor device (100) which comprises a set of technologies and components that are designed to fulfill the functions for reading and visualizing real time load data, can be used to triggering audible, sensory and visual representations based on a set of commands and scripts that are provided in the firmware of the processor (210). The user(s) can select a set of pre-programmed exercise activities designed for whole body functional training from the smartphone app (460) to provide a scoring platform for comparing scores and competing with other users on a leaderboard/scoreboard database, thereby expanding the use case of the invention to become a fitness gaming tool.

In an extended embodiment and the fact that the functionality of a leaderboard/scoreboard exists in both the smartphone app (460) and the cloud management platform (410), the database base can be accessed to provision a visually appealing physical leaderboard which will be able to display the individual users score providing for the system to be used as a competitive exercise sport as well as motivational tool for users to track and keep score. Similarly, the system can link with multi-locational venues as well as home workouts to show the scores of connected users and venues on-line.

The invention can be implemented in several combinations based on the tiered architecture of the system (FIG. 8). The hardware, application software, firmware or any combination of these can be adapted for use in an embodiment of the invention which may be physically, functionally and logically implemented in any suitable way. Indeed, the functionality may be implemented in a single unit, in a plurality of units or as part of a group of other functional units or may be physically and functionally distributed between different units.

### LIST OF COMPONENT NUMERALS

100—Form and attributes of the invention
120—Form of the invention with a rigid enclosure
130—Form of the invention with a flexible enclosure
150—Anchor buckle
200—Load sensor element
210—Load sensor processor
250—Load sensor unit
300—Resistance exercise apparatus
410—Cloud Management Platform
450—Smartwatch App
460—Smartphone App

Although the present invention has been described in connection with the specified embodiments, it is not intended to be limited to the specific form set forth herein.

Additionally, although individual features may be defined, these may possibly be advantageously combined, and the inclusion in different parts of the invention revelation does not imply that a combination of features is not feasible and/or advantageous. In addition, singular references do not exclude a plurality. Thus, references to “a”, “an”, “first”, “second” etc. do not preclude a plurality. Furthermore, reference labels shall not be construed as limiting the scope of this invention.

Numerous modifications and adaptations of the system of the present invention will be apparent to those skilled in the art, and thus it is intended by the appended claims to cover all such modifications and adaptations which fall within the true spirit and scope of this invention.

What is claimed is:

1. A load sensor device system for measuring and tracking physical exercise strength and effort and a method of operating load sensor device using a smart watch application (450), a smartphone application (460) managed using a cloud platform (410) comprises a body load sensing device (100) embedded with a miniature tension-compression load sensor element (200), characterized in that a load sensor processor (210) and a load sensor unit (250) abbreviated inside two representative embodiments, in one, a rigid enclosure(120) and a flexible enclosure (130) which attaches to representative forms of extendible and rigid resistance exercise apparatus (300) using anchor buckles (150)

2. The load sensor element (200), as claimed in claim 1, wherein the said load sensor element (200), comprises a plastic case and a flexible enclosure (130) and; anchor buckles (150) to withstand high loads which are attached to the load sensor element (200) on either side at the anchor location (260)

3. The load sensor element (200), as claimed in claim 1, wherein the said load sensor unit (250) is attached to the load sensor processor (210) and provides the tension-compression raw data readings which are processed within the firmware of the load sensor processor (210) as novel and meaningful data and exchanged via the wireless communication module within the load sensor processor (210) with the user's smartwatch application (450), user's smartphone application (460) and displayed as meaningful strength data visualization.

4. The load sensor element (200), as claimed in claim 1, wherein on powering up the load sensor element and initiating the application an initialization is performed on the load sensor processor (210); after pairing the smartphone application (460) assigns settings to the load sensor element (200) and creates a user login account with the managed cloud platform (410) to identify the specific user account with the specific device ID to ensure the readings are stored within the user profile to provide tracking analytics and smartwatch application (450) pairs with the smartphone application (460) to check the battery status and providing the user a graphical, visual and textual display of the device strength readings while the user performs resistance exercises using the resistance exercise apparatus (300).

5. The load sensor element (200), as claimed in claim 5, wherein the load sensor element (200) streams the real-time readings as they change to the connected application and management layer for storage, processing and meaningful visualization.

6. The load sensor element (200), as claimed in claim 1, wherein the said load sensor element (200) is any other type of functional equipment that produces tension compression to provide real time data readings and the open communication protocols used within the load sensor processor provide the ability to stream the data readings in open or proprietary data interchange formats making it extremely versatile.

7. The load sensor element (200), as claimed in claim 1, wherein the said smartphone application (460) when connected to the load sensor device (200) allows users to vary the timing of the exercise, the cycles as well as the representatives to suit their preferred fitness level which allows the system be useful for people of all ages and fitness levels and provides tracking information of gain or loss in strength while performing the same exercise over a period of time.

8. The load sensor element (200), as claimed in claim 1, wherein the said the load sensor device (100) is used for triggering audible, sensory and visual representations based on a set of commands and scripts that are provided in the firmware of the processor (210) and the user(s) selects a set of pre-programmed exercise activities designed for whole body functional training from the smartphone application (460) to provide a scoring platform for comparing scores and competing with other users on a leaderboard/scoreboard database.

9. The leaderboard/scoreboard (200), as claimed in claim 9, wherein the functionality of a leaderboard/scoreboard exists in both the smartphone application (460) and the cloud management platform (410), and the database base is accessed to provision a visually appealing physical leaderboard which is able to display the individual users score providing for the system to be used as a competitive exercise sport as well as motivational tool for users to track and keep score.

10. The load sensor element (200), as claimed in claim 10, wherein the said system can link with multi-locational venues as well as home workouts to show the scores of connected users and venues on-line.
